(19) United States
(12) Patent Application Publication
(10) Pub. No.: US 2013/0268023 A1
(43) Pub. Date: Oct. 10, 2013

(54) TIBIAL NERVE STIMULATION

(71) Applicant: Scott E. Jahns, Hudson, WI (US)

(72) Inventor: Scott E. Jahns, Hudson, WI (US)

(73) Assignee: AMS Research Corporation, Minnetonka, MN (US)

(21) Appl. No.: 13/991,476

(22) PCT Filed: Dec. 15, 2011
(86) PCT No.: PCT/US11/65052

§ 371 Date: Jun. 4, 2013

Related U.S. Application Data
(60) Provisional application No. 61/423,307, filed on Dec. 15, 2010.

(51) Int. Cl. A61N 1/372

(52) U.S. Cl. CPC A61N 1/37223
USPC 607/48; 607/60

(57) ABSTRACT

An implantable stimulation device comprises one or more electrodes, a receiving antenna coil, a transmitting antenna coil and a control circuit. The receiving antenna coil is in a first layer, the transmitting antenna coil is in a second layer and the control circuit is in a third layer between the first and second layers. The control circuit is configured to receive signals using the receiving antenna coil, transmit signals using the transmitting antenna coil, and deliver stimulation signals to the one or more electrodes. The receiving antenna coil, the transmitting antenna coil and the control circuit are contained in a hermetically sealed container.

### FIELD

Embodiments of the invention are directed to methods, systems and devices for stimulating the tibial nerve of a patient and, more particularly, for providing non-percutaneous stimulation of the tibial nerve for the treatment of urinary incontinence or other condition of the patient.

### BACKGROUND

Conditions such as urinary urgency, urinary frequency and urge incontinence have been treated through percutaneous tibial nerve stimulation. Conventional systems and devices for performing such a treatment require a needle electrode to be inserted near the patient's ankle to place the needle electrode adjacent the tibial nerve. The needle electrode is coupled to a hand-held stimulator that delivers electric pulses to the electrode that stimulate the sacral plexus. To achieve the best results, the patient typically must have monthly treatments with each session lasting approximately 30 minutes. Such treatments prevent the patient from being ambulatory during treatment. Additionally, there is an increased risk of infection due to the repeated needle sticks.

### SUMMARY

Embodiments of the invention are directed to methods of performing non-percutaneous stimulation of the tibial nerve of a patient. Other embodiments are directed to systems and stimulation devices for use in the method.

In one embodiment of the method, a stimulation device is implanted in the patient and one or more electrodes are implanted in the patient adjacent the tibial nerve. The stimulation device is wirelessly commanded to execute a stimulation protocol using a therapy controller located externally to the patient. A stimulation waveform is then delivered to the one or more electrodes in accordance with the stimulation protocol using the stimulation device. The tibial nerve is stimulated responsive to delivering the stimulation waveform. In one embodiment, a condition of the patient is treated responsive to the stimulation of the tibial nerve. Exemplary embodiments of the condition include urinary incontinence and urinary frequency.

One embodiment of the implantable stimulation device comprises one or more electrodes, a receiving antenna coil, a transmitting antenna coil and a control circuit. The receiving antenna coil is in a first layer, the transmitting antenna coil is in a second layer and the control circuit is in a third layer between the first and second layers. The control circuit is configured to receive signals using the receiving antenna coil, transmit signals using the transmitting antenna coil, and deliver stimulation signals to the one or more electrodes. The receiving antenna, the transmitting antenna and the control circuit are contained in a hermetically sealed container.

Other features and benefits that characterize embodiments of the invention will be apparent upon reviewing the following detailed description and associated drawings.

### BRIEF DESCRIPTION OF THE DRAWINGS

FIG. 1 is a schematic diagram of a tibial nerve stimulation system including a stimulation device and therapy controller in accordance with embodiments of the invention.

FIG. 2 is a flowchart illustrating a method of stimulating the tibial nerve of a patient in accordance with embodiments of the invention.

FIG. 3 is a simplified illustration of a therapy controller attached to a leg of a patient in accordance with embodiments of the method.

FIGS. 4 and 5 are simplified partial cross-sectional views of a stimulating device implanted in a patient in accordance with embodiments of the invention.

### DETAILED DESCRIPTION OF ILLUSTRATIVE EMBODIMENTS

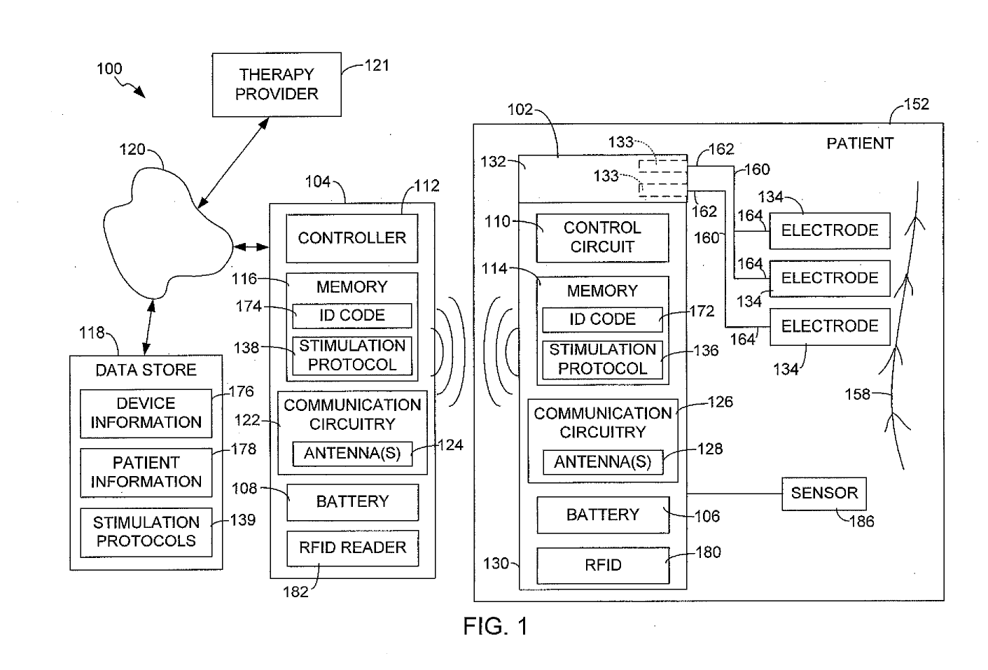

FIG. 1 is a schematic diagram of a system 100 that comprises a stimulation device 102 and a therapy controller 104 in accordance with embodiments of the invention. The stimulation device is powered by a battery 106 and is configured for implantation in a patient. The therapy controller 104 may be powered by a battery 108 or other source, such as line power.

In one embodiment, the stimulation device 102 includes a control circuit 110 and the therapy controller 104 includes a controller 112. The control circuit 110 and the controller 112 each comprise one or more processors configured to execute program instructions to perform method steps and functions described herein. In one embodiment, the program instructions that are executable by the control circuit 110 may be stored in local memory 114 of the device 102 or communicated from the therapy controller 104.

The program instructions executable by the one or more processors of the controller 112 may be stored in local memory 116 of the therapy controller 104, stored in a remote data store 118 accessible through conventional data links such as through a network 120, or communicated from a therapy provider 121 through the network 120 or other data communication link, for example.

In one embodiment, the therapy controller 104 includes communication circuitry 122 configured to communicate commands, data and/or other information to the device 102 in accordance with conventional wireless communication techniques. One embodiment of the communication circuitry 122 includes one or more antennas 124 that facilitate transmitting and/or receiving communications. In one embodiment, the communication circuitry 122 is configured to communicate with the remote therapy provider 121 and/or the remote data store 118 through conventional communication techniques, such as through the network 120.

In one embodiment, the stimulation device 102 includes communication circuitry 126 configured to communicate with the therapy controller 104 in accordance with conventional wireless communication techniques. In one embodiment, the communication circuitry 126 includes one or more antennas 128 that facilitate transmitting and/or receiving communications.

In one embodiment, the control circuit 110 and battery 106 are contained within a hermetically sealed container 130 that is configured for implantation in a patient. In accordance with other embodiments, the memory 114, one or more antennas 128 and/or other components of the device 102 are contained in the container 130, as shown in FIG. 1.

In one embodiment, the device 102 includes a header 132 attached to the container 130. In one embodiment, the header 132 includes one or more ports 133. In one embodiment, each of the ports 133 is coupled to the control circuit 110 through signal carriers that pass through the sealed container 130.

The stimulation device 102 is configured to generate electrical stimulation signals and deliver the stimulation signals to one or more electrodes 134. The electrical stimulation signals are generated by the control circuit 110 in accordance with conventional implantable stimulating devices. Embodiments of the stimulation signals include electrical waveforms that may be uniphasic or biphasic. The waveform may have a range of amplitudes, duty cycles and/or frequencies. Exemplary pulse frequencies include frequencies in the range between 5 and 200 Hz, but other frequencies outside this range may also be used. Alternatively, the waveform may comprise a decaying square wave, sinusoid or sawtooth, or have any other shape found to be suitable. Additionally, the waveform may comprise one or more bursts of short pulses. Generally, appropriate waveforms and parameters thereof are determined during the initial test period of the implantation.

In one embodiment, the stimulation signals are generated in accordance with a stimulation protocol comprising instructions that are executed by the control circuit 110. In one embodiment, the stimulation protocol defines the stimulating therapy that is to be performed by the stimulation device 102 including, for example, the electrical waveform for stimulation signals that are to be delivered to the one or more electrodes 134, the particular electrodes 134 that are to receive the stimulation signals, a duration of the stimulation signals, a frequency at which the stimulation signals are to be applied, and other information. In one embodiment, the stimulation protocol is stored in memory 114 of the device 102, as indicated at 136 in FIG. 1. In one embodiment, the stimulation protocol is stored in memory 116 of the therapy controller 104, as indicated at 138, and is communicated to the device 102 by the controller 104, such as with a command to execute a stimulation therapy as described below. The stimulation protocol may also be accessed by the therapy controller 104 from the remote data store 118 as indicated at 139, or communicated to the controller 104 from the therapy provider 121, for example.

In one embodiment, the control circuit 110 is configured to deliver the stimulation signals to the ports 133 in accordance with the stimulation protocol. In one embodiment, the control circuit 110 is configured to deliver the stimulation signals to a subset of the ports based on the stimulation protocol.

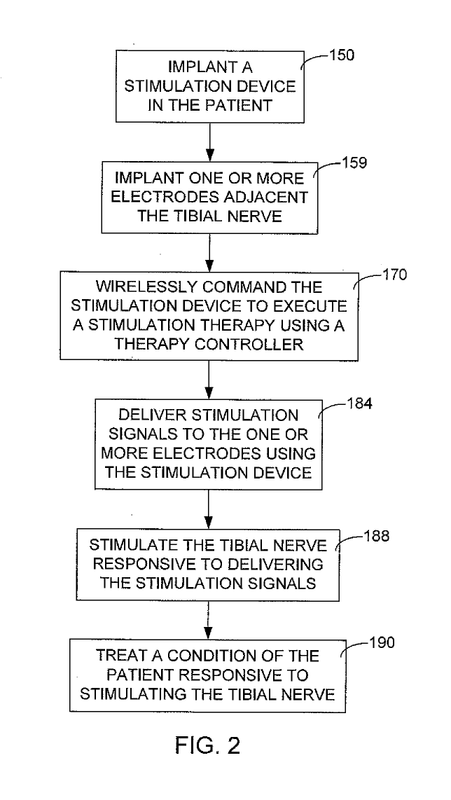

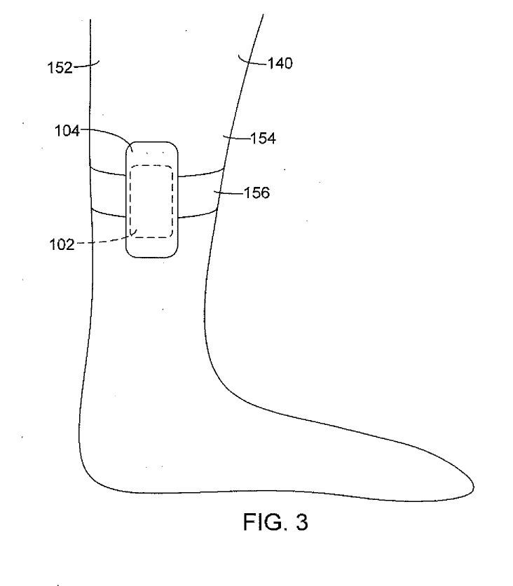

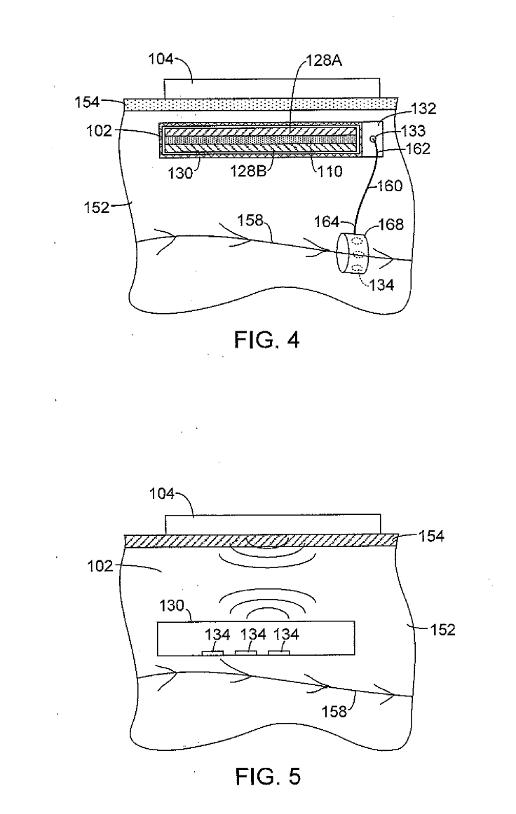

FIG. 2 is a flowchart illustrating a method of stimulating the tibial nerve of a patient in accordance with embodiments of the invention. The method will also be described with reference to FIGS. 3-5. FIG. 3 is a simplified illustration of a therapy controller 104 attached to a leg 140 of a patient in accordance with embodiments of the method. FIGS. 4 and 5 are simplified partial cross-sectional views of a stimulating device 102 implanted in a patient in accordance with embodiments of the invention.

At step 150 of the method, the stimulation device 102 is implanted in a patient 152. As used herein, the term “implanted” means that the object is surgically placed within the patient beneath the skin through an incision. In one embodiment, the device 102 is implanted in the leg 140 of the patient, such as adjacent to the ankle of the patient, as illustrated schematically in FIG. 3.

In one embodiment, the stimulation device 102 is implanted in close proximity to the skin 154 of the patient 152, as shown in FIG. 4. In one embodiment, the therapy controller 104 is also located in close proximity to the skin 154 adjacent the site of the stimulation device 102. This placement of the stimulation device 102 and the therapy controller 104 reduces the distance the communications between the devices must travel. As a result, the signals communicated between the stimulation device 102 and the therapy device 104 can be low power, which is particularly useful in conserving the energy stored in the battery 106 powering the electronics of the stimulation device 102, such as the control circuit 110. Such power conservation allows the device 102 to operate within the patient for long periods of time.

In one embodiment, the battery 106 can be charged through inductive coupling in accordance with conventional techniques. The placement of the device 102 adjacent the skin 154 facilitates such inductive charging of the battery 106.

In one embodiment, the antennas 128 of the stimulation device 102 include a receiving antenna coil 128A configured to receive the signals transmitted by the therapy controller 104. In one embodiment, the antennas 128 include a transmitting antenna coil 128B configured to wirelessly transmit data signals, which may be received by the therapy controller 104.

In one embodiment, the transmitting antenna coil 128A and the receiving antenna coil 128B are each located in separate layers, and the control circuit 110 is located in a layer between the layers of the transmitting antenna coil 128A and the receiving antenna coil 128B, as shown in FIG. 4. In one embodiment, the stimulation device 102 is implanted in the patient 152 such that the transmitting antenna coil 128A is positioned as close as possible to the skin 154, as shown in FIG. 4. This reduces the power required to communicate signals to the therapy controller 104.

In one embodiment, the therapy controller includes a strap 156 that facilitates attachment of the controller 104 to the patient 152, such as the leg of a patient 152, as shown in FIG. 3. The strap 156 operates to maintain the therapy controller 104 against the skin 154 of the patient and in close proximity to the stimulation device 102.

At step 159 of the method, one or more electrodes 134 are implanted in the patient 152 adjacent a tibial nerve 158, as shown in FIG. 1. In one embodiment, the one or more electrodes 134 are placed in contact with or close proximity to the tibial nerve 158, or a branch thereof.

In one embodiment, the electrodes 134 are coupled to the device 102 through one or more leads 160, as shown in FIG. 1. Each of the leads 160 may couple to one or more of the electrodes 134. In one embodiment, proximal ends 162 of the leads 160 couple to the ports 133 of the device 102. The stimulation signals are communicated to the ports 133 for delivery to the electrodes 134 located at a distal end 164 of the leads 160.

In accordance with another embodiment, the electrodes 134 are attached to a cuff 168, as shown in FIG. 4. In one embodiment of the implanting step 159, the cuff 168 is implanted around the tibial nerve 158, as shown in FIG. 4.

In accordance with another embodiment, the one or more electrodes 134 are formed on the container 130, as shown in FIG. 5. In one embodiment, the device 102 is placed in close proximity to the tibial nerve 158 to complete the implanting steps 120 and 124 of the method.

At step 170 of the method, the stimulating device 102 is wirelessly commanded to execute a stimulation protocol using the therapy controller 104 that is located externally to the patient 152, as illustrated in FIG. 1. In one embodiment, the command is a signal communicated wirelessly from the therapy controller 104 using the communication circuitry 122 and the one or more antennas 124, and received by the device 102 using the communication circuitry 126 and the one or more antennas 128.

In one embodiment, the command signal communicated from the therapy controller 104 is generated in accordance with program instructions executed by the one or more processors of the controller 112. In one embodiment, the command signal from the therapy controller 104 includes the stimulation protocol to be performed by the stimulation device 102, which may be retrieved from the memory 116, the data store 118 or the therapy provider 121, as mentioned above. In accordance with one embodiment, the command from the therapy controller 104 identifies one of a plurality of stimulation protocols 134 stored in the memory 114 of the stimulation device 102. In accordance with this embodiment, the control circuit 110 selects the identified stimulation protocol 134 stored in memory 114 responsive to the command signal from the therapy controller 104 for execution by the control circuit 110.

In one embodiment, the stimulation device 102 is identified by the therapy controller 104 prior to the commanding step 170. In one embodiment, this identification of the device 102 involves communicating a unique identification code 172 (FIG. 1) stored in the memory 114 of the device 102 to the therapy controller 104. In one embodiment, the controller 112 of the therapy controller 104 matches the identification code 172 received from the stimulation device 102 with a corresponding code 174 stored in the memory 116. Alternatively, the controller 104 can compare the identification code 172 to an identification code received from the therapy provider 121, or stored in the remote data store 118. In one embodiment, the comparison of the identification code 172 to the identification code 174 serves the purpose of verifying that the device 102 is a valid stimulation device, or a stimulation device that is compatible with the therapy controller 104.

In accordance with another embodiment, the identification code 172 is used to determine the stimulation protocol that is to be executed by the control circuit 110. In one embodiment, the identification code 172 is used to retrieve a stimulation protocol that is prescribed for the patient 152 associated with the identification code 172, in which the device 102 is implanted.

In accordance with another embodiment, the identification code 172 may be used to retrieve device information 176 from the therapy provider 121 or the data store 118, as shown in FIG. 1. The device information 174 can be used by the therapy controller 104 to select an appropriate stimulation protocol or retrieve other useful information about the stimulation device 102.

In accordance with another embodiment, the identification code 172 is used by the therapy controller 104 to retrieve patient information 178 from the therapy provider 121 or the remote data store 118, as shown in FIG. 1.

In one embodiment, the identification code 172 is stored in a radio frequency identification (RFID) circuit 180 attached to or contained within the container 130 of the device 102. In one embodiment, the therapy controller 104 includes an RFID reader 182 that is configured to extract the identification code 172 from the RFID circuit 180.

At step 184 of the method, the stimulation device 102 delivers stimulation signals to the one or more electrodes 134 in accordance with the stimulation protocol. In one embodiment, the one or more processors of the control circuit 110 execute the instructions of the stimulation protocol to cause the generation and delivery of electrical stimulation signals to the one or more electrodes 134.

In one embodiment of the stimulating step 184, the stimulation waveform or signal generated by the control circuit 110 is delivered to a subset of the electrodes 134. In one embodiment, the stimulation device 102 includes more than three electrodes 134 and the stimulation signal is delivered to a subset of the electrodes 134 that includes two or more of the electrodes 134. In one embodiment, this delivery of the stimulation signal to a subset of the electrodes 134 involves delivering the stimulation signal to a subset of the available ports 133 to which the electrodes 134 are electrically coupled.

In one embodiment, the subset of the electrodes that are to receive the stimulation signal are identified in the stimulation protocol executed by the control circuit 110, such as the stimulation protocol 134 contained in the memory 114 of the device, for example. Alternatively, the subset of the electrodes 134 that are to receive the stimulation signal may be identified in the command signal from the therapy controller 104.

In one embodiment, the subset of the electrodes 134 are determined through an interrogation of the electrodes 134 by the stimulation device 102, or during a testing phase of the implantation of the electrodes 134 by an external controller. In one embodiment, such a testing phase involves the delivery of stimulation signals to each of the electrodes 134, or groups of the electrodes 134, to determine which of the electrodes 134 provides the most effective stimulation of the tibial nerve 158.

In one embodiment, this interrogation of the electrodes 134 may be determined using a suitable sensor 186. In one embodiment, the sensor 186 provides an electromyographic signal back to the control circuit 110 that is indicative of the stimulation therapy performed by the device 102. The signal is analyzed by the control circuit 110 to determine which electrodes 134 are best suited for delivering the stimulation signal.

At step 188 of the method, the tibial nerve 158 is then stimulated responsive to the delivery of the stimulation signals to the one or more electrodes 134. In one embodiment, a condition of the patient is treated responsive to the stimulation of the tibial nerve 158, at 190. Embodiments of the condition of the patient include urinary incontinence, such as urge incontinence. Other exemplary embodiments of the condition include urinary frequency.

Although the present invention has been described with reference to preferred embodiments, workers skilled in the art will recognize that changes may be made in form and detail without departing from the spirit and scope of the invention.

1. A method of stimulating a tibial nerve of a patient comprising:
implanting a stimulation device in the patient;
implanting one or more electrodes in the patient adjacent the tibial nerve;
wirelessly commanding the stimulation device to execute a stimulation protocol using a therapy controller located externally to the patient;
delivering a stimulation waveform to the one or more electrodes in accordance with the stimulation protocol using the stimulation device; and
stimulating the tibial nerve responsive to delivering a stimulation waveform.

2. The method of claim 1, wherein implanting a stimulation device in the patient comprises implanting the stimulation device in the leg of the patient.

3. The device of claim 2, further comprising, attaching the therapy controller to the leg of the patient adjacent the stimulation device prior to wirelessly commanding the stimulation device to execute a stimulation protocol.

4. The device of claim 2, wherein implanting a stimulation device in the patient comprises implanting the stimulation device in close proximity to the skin of the patient.

5. The method of claim 1, wherein implanting one or more electrodes comprises implanting a plurality of electrodes.

6. The method of claim 5, wherein delivering a stimulation waveform comprises delivering a stimulation waveform to a subset of the electrodes.

7. The method of claim 6, further comprising identifying the subset of the electrodes in memory of the stimulation device or in the stimulation protocol.

8. The method of claim 1, wherein:
the method comprises providing a cuff including the one or more electrodes; and
implanting one or more electrodes in the patient adjacent the tibial nerve comprises implanting the cuff around the tibial nerve.

9. The method of claim 1, further comprising identifying the stimulation device prior to commanding the stimulation device to execute a stimulation protocol using the therapy controller.

10. The method of claim 9, wherein identifying the stimulation device comprises communicating a unique identification code between the stimulation device and the therapy device.

11. The method of claim 1, further comprising treating urinary incontinence in response to stimulating the tibial nerve.

12. The method of claim 1, further comprising receiving stimulation therapy information from a remote therapy provider using the therapy controller.

13. The method of claim 12, wherein the stimulation therapy information includes the stimulation protocol.

14. An implantable stimulation device comprising:
one or more electrodes;
a transmitting antenna coil in a first layer;
a receiving antenna coil in a second layer;
a control circuit in a third layer between the first and second layers, wherein the control circuit is configured to receive signals using the receiving antenna, transmit signals using the transmitting antenna, and deliver stimulation signals to the one or more electrodes;
a hermetically sealed container containing the receiving antenna coil, the transmitting antenna coil and the control circuit.

15. The device of claim 14, further comprising a radio frequency identification circuit including memory having a unique identification.

16. The device of claim 14, further comprising a header attached to the container, the header including one or more ports each configured to couple to an electrode lead, wherein the control circuit is configured to deliver the stimulation signals to the one or more ports.

17. The device of claim 16, further comprising one or more electrode leads each having a proximal end coupled to one of the ports and a distal end coupled to at least one of the electrodes.

18. The device of claim 16, wherein:
the device comprises a plurality of the electrodes; and
the control circuit is configured to direct the stimulation signal to a subset of the electrodes.

19. The device of claim 16, wherein:
the header comprises a plurality of the ports; and
the control circuit is configured to direct the stimulation signals to a subset of the ports.

20. The device of claim 16, wherein the one or more electrodes are formed on the container.
